(19) United States
(12) Patent Application Publication
(10) Pub. No.: US 2015/0015878 A1
(43) Pub. Date: Jan. 15, 2015

(54) RAMAN SPECTROSCOPIC ANALYZING APPARATUS

(71) Applicant: SHIMADZU CORPORATION, Kyoto (JP)

(72) Inventor: Naoji MORIYA, Kyoto (JP)

(21) Appl. No.: 14/315,815

(22) Filed: Jun. 26, 2014

(30) Foreign Application Priority Data
Jul. 11, 2013 (JP) 2013-145359

(51) Int. Cl. G01J 3/44; G01N 33/00; G01J 3/02

(52) U.S. Cl. CPC G01J 3/4412; G01J 3/0216; G01N 33/0009
USPC 356/301

(57) ABSTRACT

A Raman spectrometry apparatus comprises a condensing unit that condenses a light flux emitted from a light source to a prescribed position in a sample; a retroreflective unit that is disposed opposite to the condensing unit with reference to the sample; and a detecting unit that detects scattering light released from the prescribed position in the sample. The retroreflective unit again condenses the light flux having transmitted through the sample to become incident on the retroreflective unit to the prescribed position, irrespective of any change in disposition of the retroreflective unit. The retroreflective unit has at least one corner cube prism.

### TECHNICAL FIELD

The present invention relates to a component analyzing apparatus by means of Raman scattering light. In particular, the present invention relates to a Raman spectroscopic analyzing apparatus that reflects a light flux having transmitted through a sample such that the sample is again irradiated with the light flux.

### BACKGROUND ART

An apparatus that analyzes component contained in a sample by means of Raman spectrometry includes a light source that emits light with which a sample is irradiated (excitation light), an incident optical system that condenses the excitation light to irradiate the sample therewith, a spectral optical system that condenses Raman scattering light generated by interaction between light and a substance contained in the sample and that spectrally disperses the light, and a detector that detects the light having undergone the wavelength separation at the spectral optical system.

By plotting the intensity of light from a sample such that the horizontal axis indicates the wavelength and the vertical axis indicates the intensity, a Raman scattering spectrum can be obtained on both sides in wavelength with the excitation light. The long wavelength side relative to the excitation light wavelength is referred to as the Stokes line, and the short wavelength side relative to the excitation light wavelength is referred to as the anti-Stokes line.

The energy that corresponds to the difference between the wavelength of the excitation light and the wavelength of the Stokes line or that of the anti-Stokes line reflects the energy of natural vibration of a molecule. Accordingly, by obtaining the energy thereof, the substance contained in the sample can be specified. Further, from the intensity of the Stokes line or anti-Stokes line appearing in the Raman scattering spectrum, the quantity of the substance corresponding to such Stokes line or anti-Stokes line can be determined.

In general, the intensity of Raman scattered light released from gas is very weak. In consideration of this point, there is disclosed a gas component analyzing apparatus that causes light having transmitted through a sample gas in a sample chamber to be again incident on the sample, such that a greater amount of Raman scattered light is produced (S. C. Eichmann, M. Weschta, J. Kiefer, T. Seeger, and A. Leipertz, “Characterization of a fast gas analyzer based on Raman scattering for the analysis of synthesis gas”, Rev. Sci. Instrum. 81, 125104 (2010)) (Non-Patent Literature 1).

The substantial structure of a gas component analyzing apparatus 100 disclosed in Non-Patent Literature 1 is shown in FIG. 1.

Excitation light emitted from a light source 101 is reflected off a mirror 102, and is condensed by a lens 103 to a prescribed position in a sample chamber 110.

The excitation light having focused at the prescribed position of a sample gas passes through a light transmitting window 112 and exits from the sample chamber 110. Then, a lens 104 collimates the excitation light, and the excitation light becomes incident on a right-angle prism 105. The traveling direction of the light is reflected by the right-angle prism 105, and the light is condensed by the lens 104 again to the prescribed position in the sample chamber 110.

The excitation light passed through the sample gas and exits from a light transmitting window 111. The light is reflected off a mirror 106 and directed to a beam damper 107.

Raman scattered light generated from the sample gas exits from the sample chamber 110 through a light transmitting window 113 provided at a position being substantially perpendicular to the optical axis of the incident light flux, and attaines on a detecting unit 109 through a lens 108.

When a Raman spectroscopic analyzing apparatus is affected by any vibrations or temperature variations, in some cases, positions with each optical element may be changed. In connection with the apparatus described above, when the right-angle prism 105 or the retaining mechanism thereof is slightly displaced whereby the incident angle of excitation light is changed, the reflected light from the right-angle prism 105 will be slightly different from an designed direction. As a result, the output light from the right angle prism 105 is not condensed to the prescribed position in the sample chamber 110. Then, the intensity of the Raman scattering light generated from the prescribed focusing position with the excited light in the sample is reduced, whereby the amount of Raman scattering light received by the detecting unit 109 is reduced. As a result, the precision in determining the component contained in the sample gas or the concentration of the component disadvantageously becomes poor.

Here, though the description has been given of an exemplary case where the sample is gas, the same problem occurs also with the case where the sample is liquid or solid.

An object of the present invention is to provide a Raman spectroscopic analyzing apparatus with which a reduction in the intensity of Raman scattering light detected by a detecting unit does not occur even when a light reflective member is displaced.

### SUMMARY OF THE INVENTION

A Raman spectrometry apparatus comprises: a condensing unit that condenses a light flux emitted from a light source to a prescribed position in a sample; a retroreflective unit that is disposed opposite to the condensing unit with reference to the sample; and a detecting unit that detects scatteringed light released from the prescribed position in the sample.

The retroreflective unit again condenses the light flux having transmitted through the sample to become incident on the retroreflective unit to the prescribed position, irrespective of any change in disposition of the retroreflective unit.

The retroreflective unit has at least one corner cube prism.

The retroreflective unit has a cat's-eye system.

The retroreflective unit has an optical element structured by at least one bead being disposed in an in-plane direction that is perpendicular to an optical axis of the light flux becoming incident on the retroreflective unit.

A gas component analyzing apparatus comprises the Raman spectrometry apparatus.

With the Raman spectroscopic analyzing apparatus according to the present invention, a light flux emitted from a light source is condensed to a prescribed position in the sample by the condensing unit. After having transmitted through the sample, the light flux becomes incident on the retroreflective unit while diverging. The retroreflective unit returns the light flux in the incident direction irrespective of the angle of incidence of the light flux incident on the retroreflective unit. Accordingly, even when the retroreflective unit is displaced, the light flux output from the retroreflective unit reversely proceeds along the optical path of the incident light flux, and is condensed to the same position as the incident light flux, that is, to the prescribed position in the sample. Accordingly, the intensity of the Raman scattering light detected by the detecting unit will not be reduced.

The scattering light generated from the sample is incident on the detecting unit disposed at the position away from the optical path of the light flux condensed to the prescribed position in the sample (for example, at the position being perpendicular to the optical path of the light flux with which the sample is irradiated), and a Raman scattering spectrum is created by any appropriate analyzing apparatus. Then, by analyzing the Raman scattering spectrum, the substance contained in the sample is specified and the quantity there of is determined.

In connection with the Raman spectroscopic analyzing apparatus of the present invention, since the retroreflective unit that reflects the light having transmitted through a sample is included, even when the retroreflective unit is displaced to some extent, the light flux output from the retroreflective unit reversely proceeds along the optical path of the incident light flux, and is condensed again to the prescribed position in the sample. Accordingly, even when the retroreflective unit is displaced to some extent, the position of the Raman scattering light released from the sample does not change. Therefore, the detected intensity of the Raman scattering light will not be reduced.

### BRIEF DESCRIPTION OF THE DRAWINGS

FIG. 1 is a substantial part configuration diagram of a conventional Raman spectrometry apparatus;

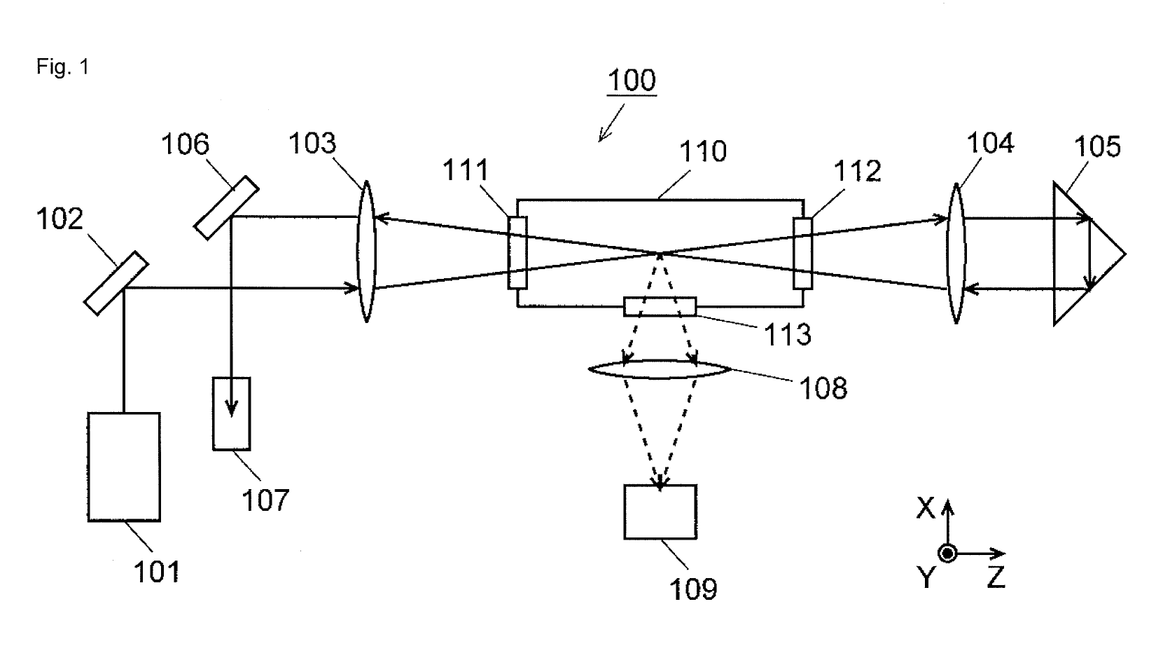

FIG. 2 is a schematic configuration diagram of a Raman spectroscopic analyzing apparatus according to one embodiment of the present invention;

FIG. 3 shows a simulation result of the case where a reflective unit of the conventional Raman spectrometry apparatus is used;

FIG. 4 shows another simulation result of the case where the reflective unit of the conventional Raman spectrometry apparatus is used;

FIG. 5 shows a simulation result of the case where a retroreflective unit of the Raman spectroscopic analyzing apparatus according to one embodiment of the present invention is used;

FIG. 6 shows another simulation result of the case where the retroreflective unit of the Raman spectroscopic analyzing apparatus according to one embodiment of the present invention is used;

FIG. 7 is a diagram illustrating a first variation of the retroreflective unit used in the Raman spectroscopic analyzing apparatus according to the embodiment;

FIG. 8 is a diagram illustrating a second variation of the retroreflective unit used in the Raman spectroscopic analyzing apparatus according to the embodiment; and

FIG. 9 is another diagram illustrating the second variation of the retroreflective unit used in the Raman spectroscopic analyzing apparatus according to the embodiment.

### DETAILED DESCRIPTION OF THE EXEMPLARY EMBODIMENT

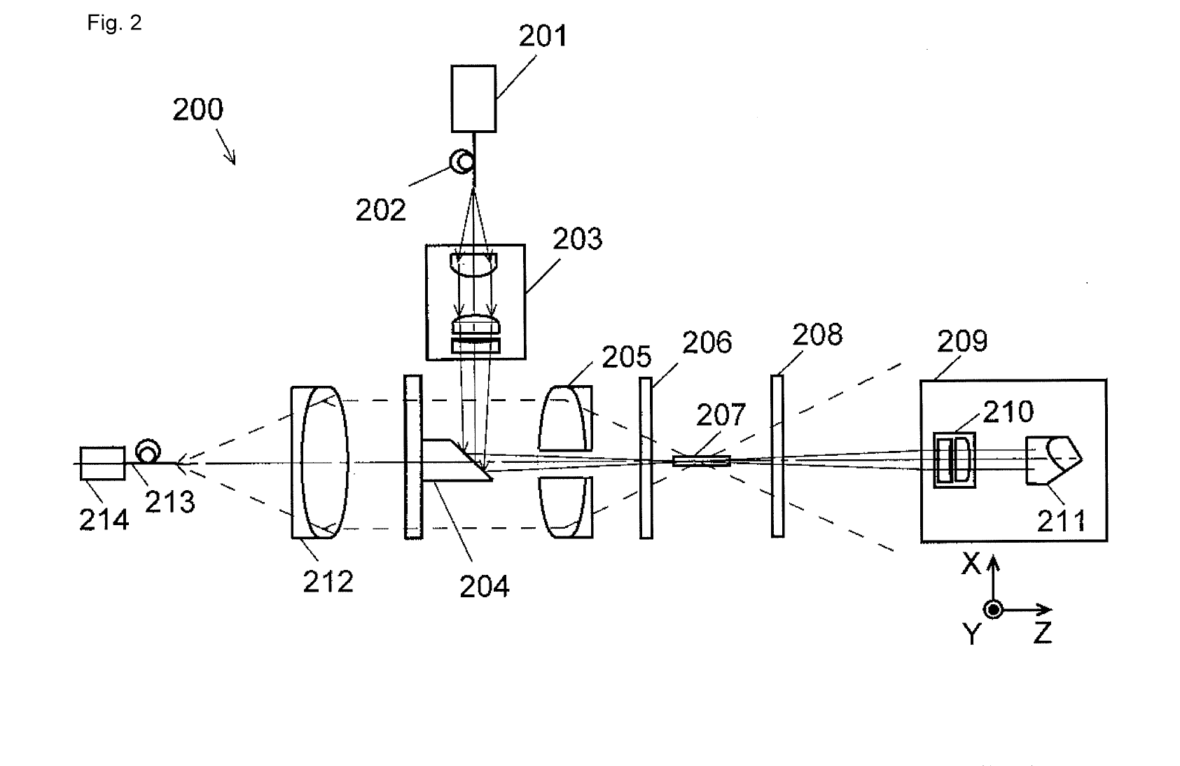

With reference to FIG. 2, a description will be given of a Raman spectroscopic analyzing apparatus according to one embodiment of the present invention. A Raman spectroscopic analyzing apparatus 200 according to the present embodiment includes a laser light source 201 that emits excitation light, an optical fiber 202 that guides the excitation light to a condensing optical system 203, a surface mirror-type combining optical system 204 that reflects the excitation light having transmitted through the condensing optical system. 203 and that aligns the optical axis of the light flux directed to a sample 207 through a sample window 206 and the optical axis of Raman scattering light from the sample 207 with each other, a collimating lens 205 that guides the Raman scattering light to the detector 214, a condenser lens 212, an optical fiber 213, and a retroreflective unit 209 that reflects the excitation light having transmitted through the sample 207 and a sample window 208 to be condensed again to the sample 207. The surface mirror-type combining optical system 204 is made up of a back plate being transparent to Raman scattered light, and a mirror fixed to the back plate. Further, the collimating lens 205 has an opening on the optical path of the excitation light so as not to influence the excitation light.

The laser light source 201 is a light source that generates visible light, and a solid-state laser such as a YAG laser or an YVO4 laser, or a gas laser such as an Ar laser is used.

The excitation light emitted from the laser light source 201 transmits through the condensing optical system 203 via the optical fiber 202. Then, the excitation light reflects off the mirror included in the surface mirror-type combining optical system 204, and enters inside the sample chamber in the direction being perpendicular to the sample window 206. In the present embodiment, the condensing optical system 203 and the surface mirror-type combining optical system 204 correspond to a condensing unit of the present invention.

The excitation light is condensed to a prescribed position within the sample 207, which is the focus of the condensing optical system 203, and excites the sample 207 at that position. The excitation light having transmitted through the sample 207 exits from the sample chamber passing through the sample window 208 located opposite to the sample window 206 with reference to the sample 207, and becomes incident on the retroreflective unit 209 having a lens system 210 and a corner cube prism 211. The retroreflective unit 209 is disposed opposite to the condensing optical system 203 and the surface mirror-type combining optical system 204 with reference to the sample 207 on the optical path of the excitation light.

The excitation light having transmitted through the sample 207 is converted into collimated light by the lens system 210, and reflects off the corner cube prism 211. The corner cube prism 211 is also referred to as a trihedral reflector. The corner cube prism 211 has three inner total reflection faces being perpendicular to one another. The total reflection of the three inner total reflection faces realizes the function of returning the light in the incident direction by 180° irrespective of the direction of light incident on the corner cube prism 211 (the retroreflective function). Accordingly, even when disposition of the corner cube prism. 211 structuring the reflective member is changed by application of external force such as vibrations or temperature variations, the excitation light having transmitted through the sample 207 and became incident on the corner cube prism 211 is again condensed to the prescribed position within the sample 207 being the focus of the condensing optical system 203.

When the sample 207 is irradiated with the excitation light, scattering light such as Rayleigh scattering light or Raman scattering light is generated from the sample 207. The backscattered Raman scattered light is converted into collimated light by the collimating lens 205. Thereafter, the Raman scattering light transmits through the back plate of the surface mirror-type combining optical system 204, and is detected by the detector 214 via the condenser lens 212 and the optical fiber 213. As the detector 214, a photoelectric conversion device such as a CCD detector is used.

From the signal detected by the detector 214, a Raman scattering spectrum is created and displayed as appropriate by the analyzing apparatus. Analysis of information of the Raman scattering spectrum such as Raman shift, Raman scattering light intensity, and spectrum width makes it possible to specify the substance contained in the sample 207 and the quantity of the substance.

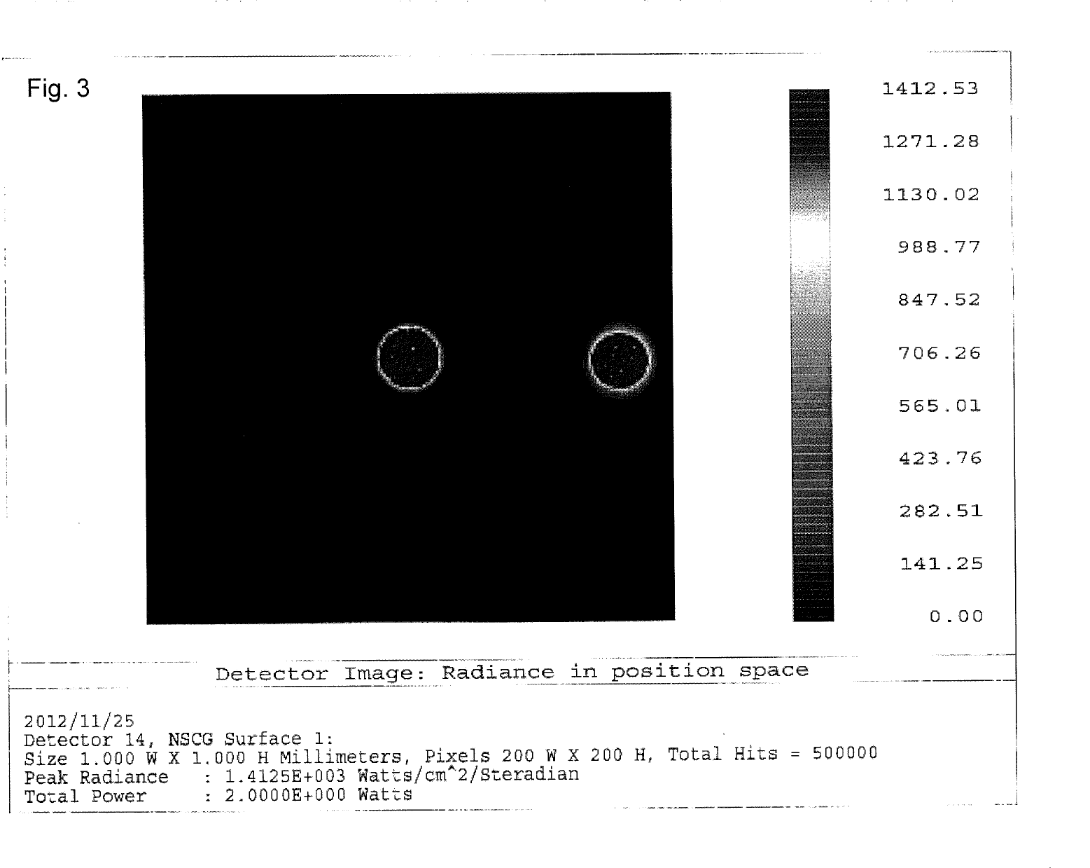

FIGS. 3 to 6 show the result of displacement simulations as to the focus of the excitation light reflected off the reflective unit (hereinafter referred to as the “reflective focus”) for each of the conventional Raman spectrometry apparatus including the right-angle prism as the reflective unit and the Raman spectroscopic analyzing apparatus including the retroreflective unit 209 according to the present embodiment.

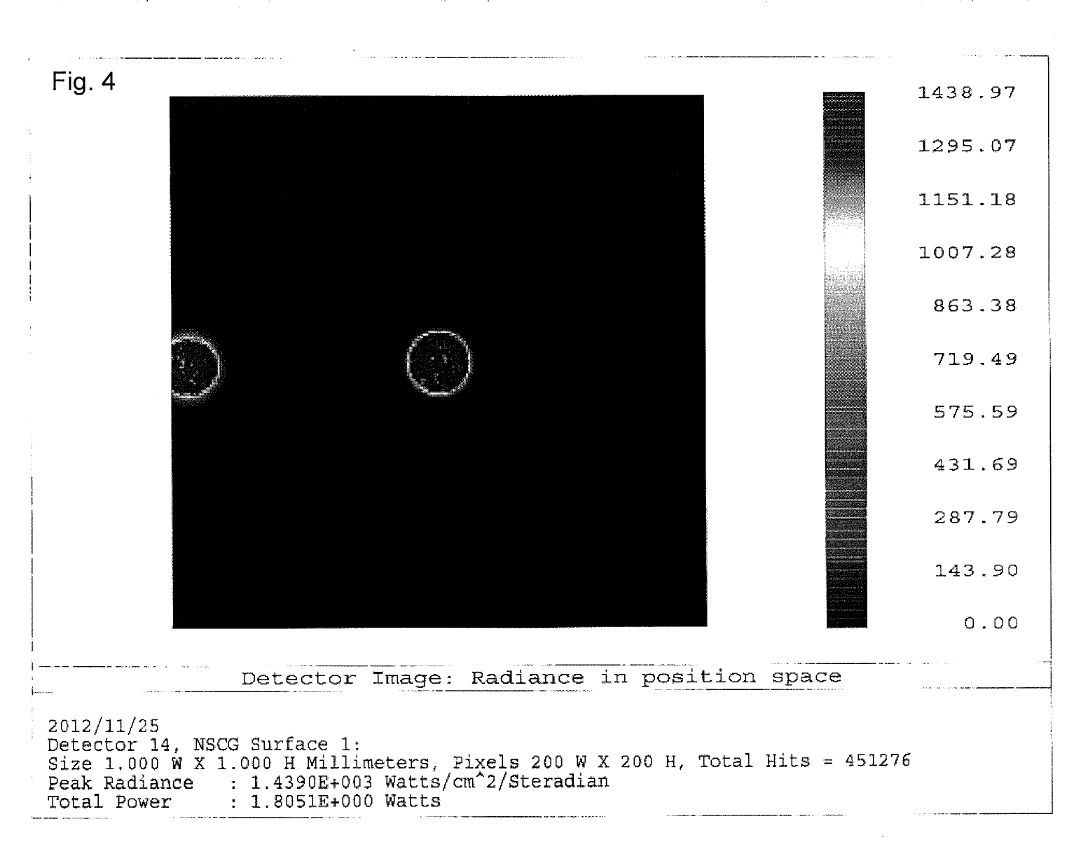

FIG. 3 shows a simulation result of the case where the reflective unit of the conventional Raman spectrometry apparatus is displaced by 0.2 mm in X-axis direction (see FIG. 2), which is one direction within a plane being perpendicular to the optical axis (Z axis) of the excitation light incident on the reflective unit, and FIG. 4 shows a simulation result of the case where the conventional reflective unit is tilted by 0.2° in Y-axis direction (i.e., the case where the reflective unit is rotated by 0.2° about X axis). FIGS. 3 and 4 each show a range of 1 mm square.

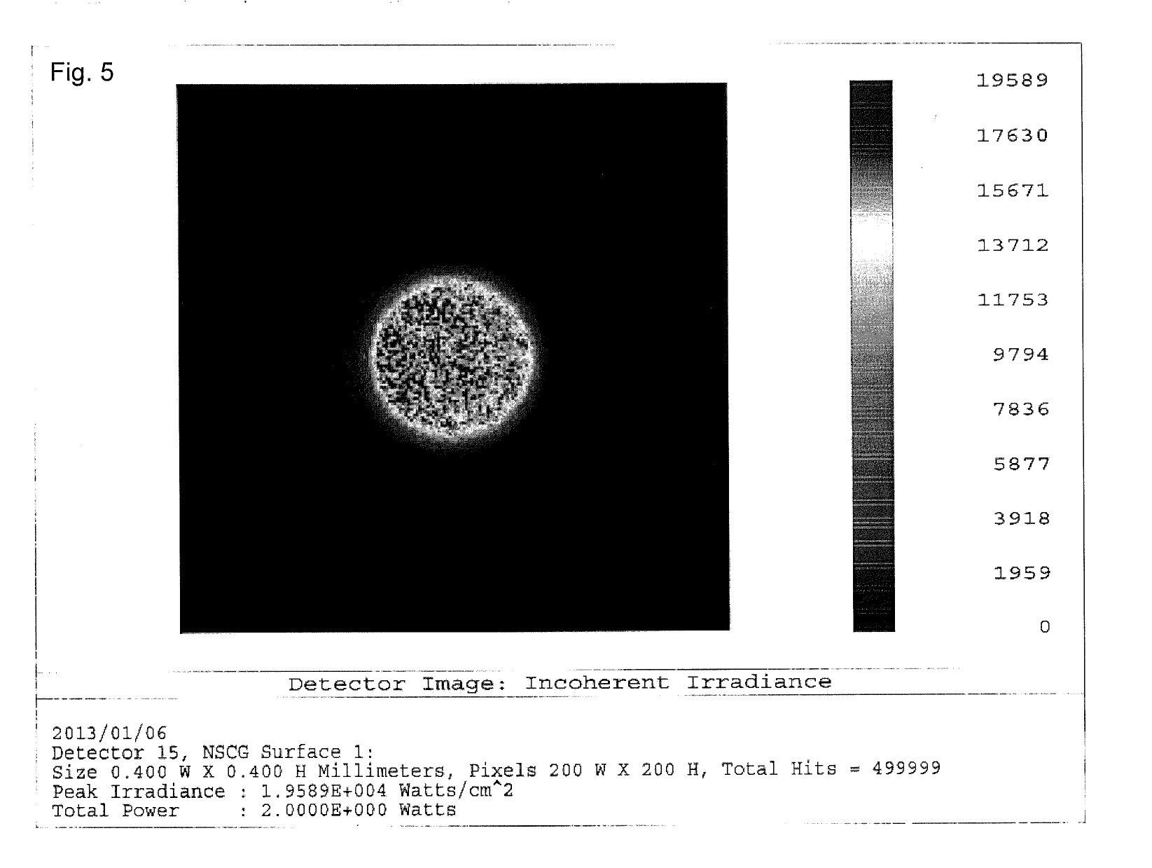

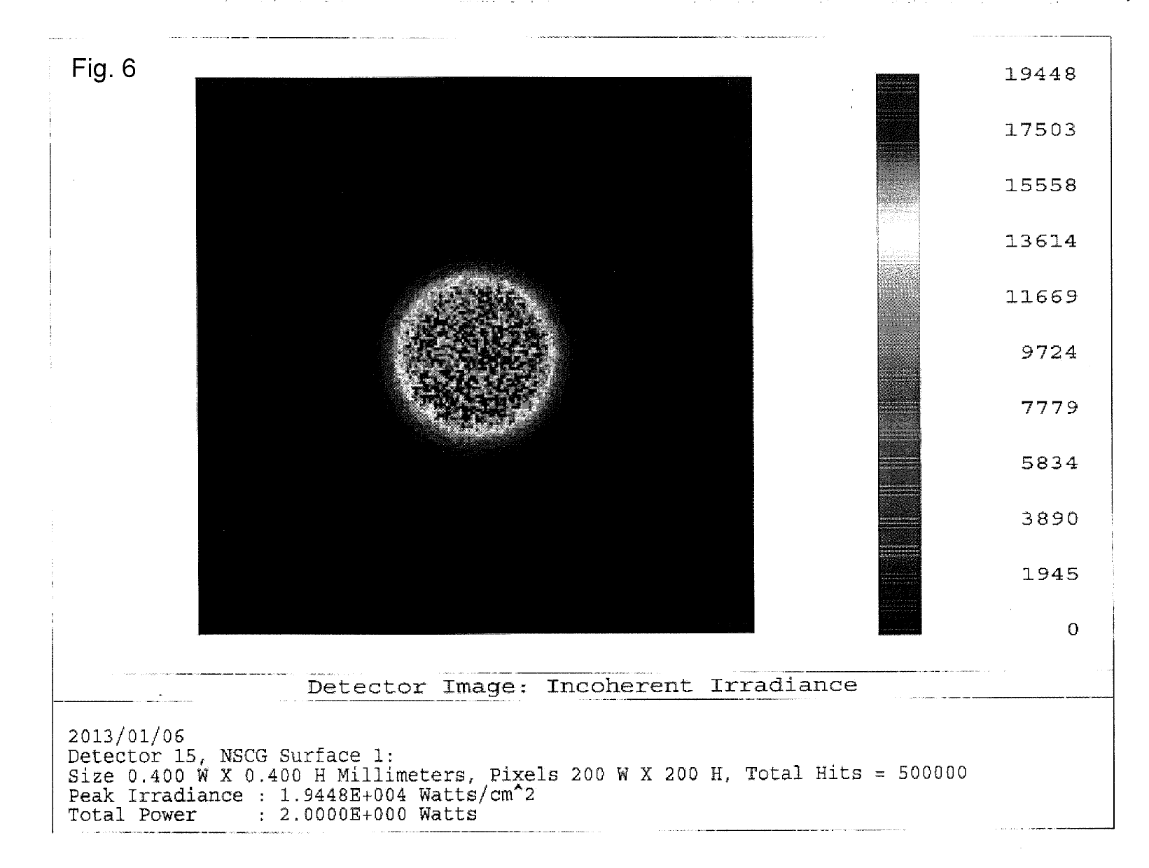

Further, FIG. 5 shows a simulation result of the case where the retroreflective unit 209 according to the present embodiment is displaced by 1 mm in X-axis direction, and FIG. 6 shows a simulation result of the case where the retroreflective unit 209 according to the present embodiment is tilted by 2° in Y-axis direction (i.e., the case where the retroreflective unit 209 is rotated by 2° about X axis) (more specifically, the case where the rotation angle about the axis that passes through the incident point of excitation light on the lens system 210 of the retroreflective unit 209 is 2°). FIGS. 5 and 6 each show a range of 0.4 mm square.

With reference to the simulation result shown in FIG. 3, with the conventional reflective unit, even in the case where the reflective unit is displaced by 0.2 mm. in X-axis direction, the position of the reflective focus is largely displaced from the position of the focus of excitation light with which the sample is firstly irradiated (hereinafter referred to as the “incident focus”) (the center in FIG. 3). Further, with reference to the simulation result shown in FIG. 4, with the conventional reflective unit, even in the case where the reflective unit is tilted by 0.2° in Y-axis direction, the reflective focus position is largely displaced from the incident focus position at the center in FIG. 4. On the other hand, with reference to the simulation result shown in FIG. 5, with the retroreflective unit according to the present embodiment, even in the case where the retroreflective unit is displaced in X-axis direction by 1 mm, which is five times as great as the value of the conventional example, the reflective focus position is overlaid on the incident focus position at the center in FIG. 5. Further, with reference to the simulation result shown in FIG. 6, with the retroreflective unit according to the present embodiment, even in the case where the retroreflective unit is tilted in Y-axis direction by 2°, which is ten times as great as the value of the conventional example, the reflective focus position is overlaid on the incident focus position at the center in FIG. 6.

Note that, while not shown, simulations were performed under the conditions in which X axis and Y axis of the above-described simulation conditions are replaced by each other. That is, simulations were also performed as to the case where the retroreflective unit 209 of the present embodiment is displaced by 1 mm in Y-axis direction, and the case where the retroreflective unit 209 is tilted by 2° in X-axis direction (i.e., the case where the retroreflective unit 209 is rotated by 2° about Y axis) (more specifically, the case where the rotation angle about the axis that passes through the incident point of excitation light on the lens system 210 of the retroreflective unit 209 is 2°). As a result, the reflective focus position is overlaid on the incident focus position.

Further, simulations were also performed as to each of the case where the rotation position of the retroreflective unit of the present embodiment is a prescribed position in the sample 207 and the case where the rotation position of the retroreflective unit is the position where the excitation light is incident on the condensing optical system 203, under the condition that the retroreflective unit 209 is tilted by 2° in X-axis direction (i.e., the case where the retroreflective unit 209 is rotated by 2° about Y axis) and the condition that the retroreflective unit 209 is tilted by 2° about Y-axis direction (i.e., the case where the retroreflective unit 209 is rotated by 2° about X axis). As a result, the reflective focus position is overlaid on the incident focus position in each of the cases.

These simulation results show that, in contrast to the reflective unit of the conventional Raman spectrometry apparatus, use of the retroreflective unit of the Raman spectroscopic analyzing apparatus according to one embodiment of the present invention can realize an excellent retroreflective function which is not influenced by displacement of the reflective unit.

As described above, even when the retroreflective unit 209 is displaced to some extent, the light flux reflected off the retroreflective unit is condensed to the incident focus position. Therefore, it becomes possible to prevent a reduction in the detected intensity of Raman scattering light that is generated at the incident focus position.

The present invention is not limited to the embodiment described above, and any modification, addition, improvement and the like can be made within the range not deviating from the gist of the invention.

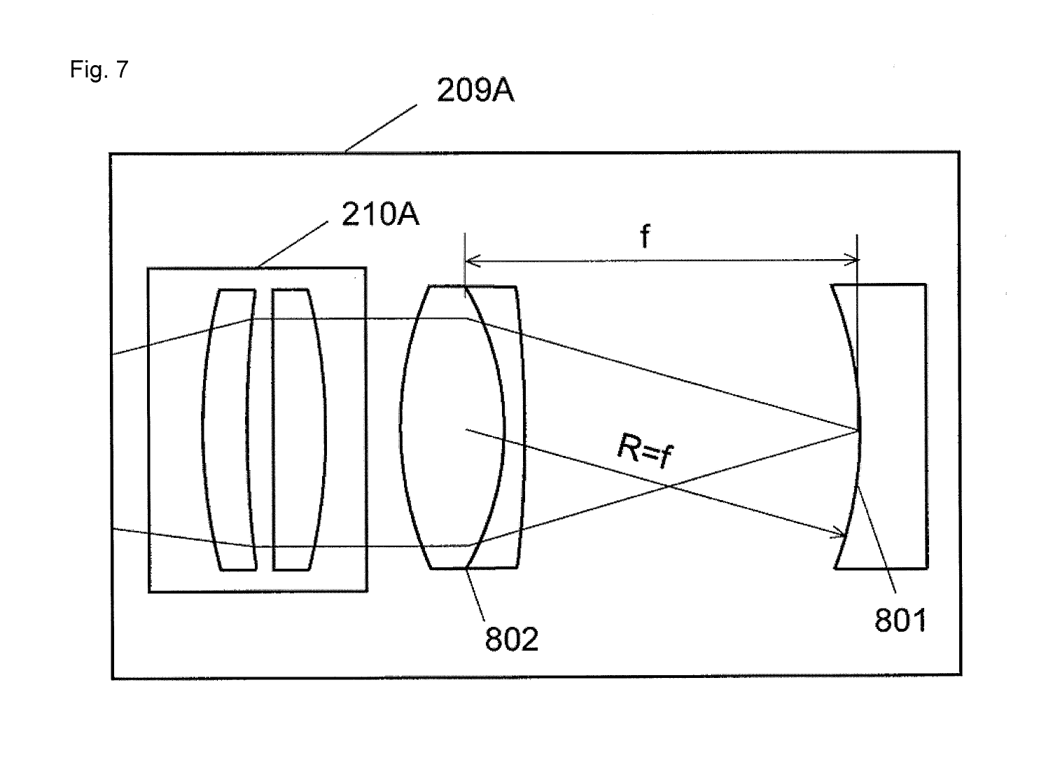

With reference to FIG. 7, a description will be given of a first variation of the retroreflective unit of the Raman spectroscopic analyzing apparatus according to the present embodiment. A retroreflective unit 209A shown in FIG. 7 includes, similarly to the retroreflective unit 209 shown in FIG. 2, a lens system 210A that converts light into collimated light. The retroreflective unit 209A further includes a cat's-eye system made up of a concave mirror 801 and a convex lens 802 disposed to be away from the concave mirror 801 by the radius of curvature R of the concave mirror 801. Here, the convex lens 802 and the concave mirror 801 are disposed such that the focal length f of the convex lens 802 and the radius of curvature R of the concave mirror 801 are equalized with each other.

The cat's-eye system is an optical system having a retroreflective function. Accordingly, even when the disposition of the concave mirror 801 structuring the retroreflective unit 209A is changed by application of external force such as vibrations or temperature variations, the excitation light having transmitted through the sample 207 and became incident on the concave mirror 801 is again condensed to a prescribed position within the sample 207 being the focus of the condensing optical system 203. Thus, it becomes possible to prevent a reduction in the intensity of Raman scattering light.

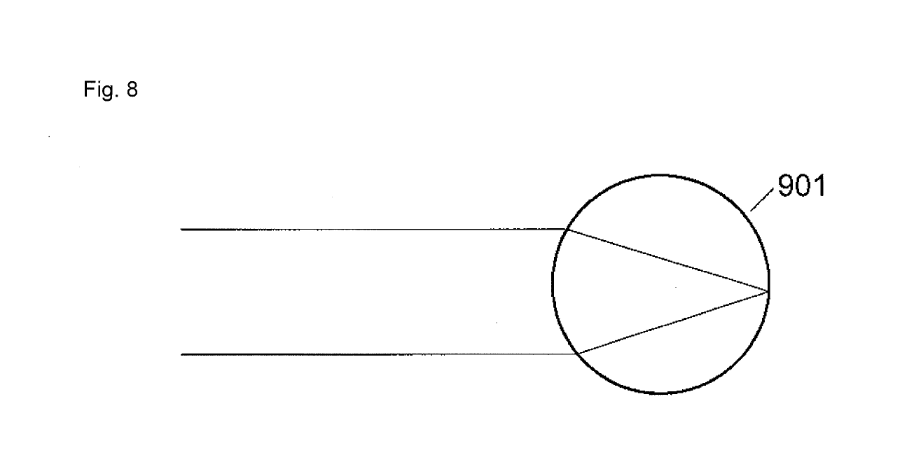

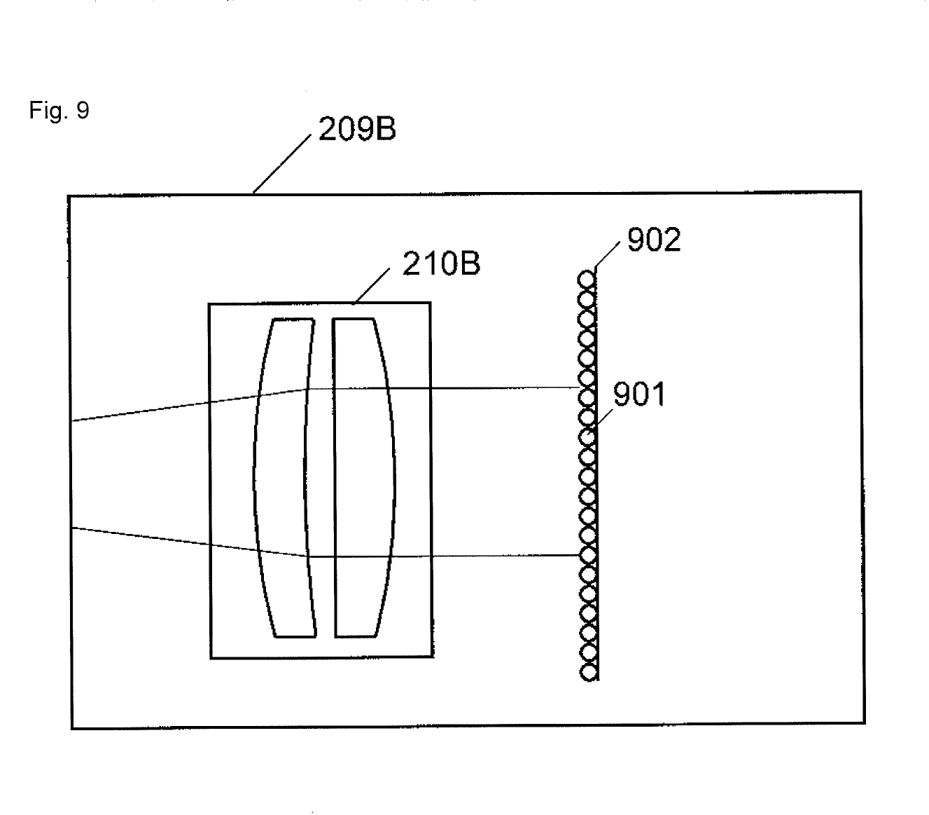

With reference to FIGS. 8 and 9, a description will be given of a second variation of the retroreflective unit 209 of the Raman spectroscopic analyzing apparatus according to the present embodiment. A bead 901 shown in FIG. 8 has a function of refracting and reflecting incident collimated light, such that the collimated light returns to the incident route. A retroreflective unit 209B shown in FIG. 9 includes, similarly to the retroreflective unit 209 shown in FIG. 2, a lens system 210B that converts light into collimated light. The retroreflective unit 209B further includes an optical element 902 in which a multitude of beads 901, one of which is shown in FIG. 8, are juxtaposed to one another in the in-plane direction being perpendicular to the optical axis of the light flux becoming incident on the retroreflective unit 209B.

The optical element 902 in which a multitude of beads 901 are juxtaposed to one another has a retroreflective function of reflecting the collimated light portion becoming incident on each bead 901 as shown in FIG. 8, and as a whole reflecting the collimated light converted by the lens system 210B. Accordingly, even when disposition of the optical element 902 structuring the retroreflective unit 209B is changed by application of external force or temperature variations, the excitation light having transmitted through the sample 207 and became incident on the optical element 902 is again condensed to a prescribed position within the sample 207 being the focus of the condensing optical system 203. Thus, it becomes possible to prevent a reduction in the detected intensity of Raman scattered light.

What is claimed is:

1. A Raman spectrometry apparatus comprising:
a condensing unit that condenses alight flux emitted from a light source to a prescribed position in a sample;
a retroreflective unit that is disposed opposite to the condensing unit with reference to the sample; and
a detecting unit that detects scattering light released from the prescribed position in the sample.

2. The Raman spectrometry apparatus according to claim 1, wherein
the retroreflective unit again condenses the light flux having transmitted through the sample to become incident on the retroreflective unit to the prescribed position, irrespective of any change in disposition of the retroreflective unit.

3. The Raman spectrometry apparatus according to claim 1, wherein
the retroreflective unit has at least one corner cube prism.

4. The Raman spectrometry apparatus according to claim 1, wherein
the retroreflective unit has a cat's-eye system.

5. The Raman spectrometry apparatus according to claim 1, wherein
the retroreflective unit has an optical element structured by at least one bead being disposed in an in-plane direction that is perpendicular to an optical axis of the light flux becoming incident on the retroreflective unit.

6. A gas component analyzing apparatus comprising the Raman spectrometry apparatus according to claim 1.
